(19) United States
(12) Patent Application Publication
(10) Pub. No.: US 2026/0269083 A1
(43) Pub. Date: Sep. 10, 2026

(54) ASSISTING CLINICAL DECISION-MAKING IN DRUG THERAPY FOR ACUTE HEART FAILURE PATIENTS

(71) Applicants: NTT RESEARCH, INC., Sunnyvale, CA (US); NATIONAL CEREBRAL AND CARDIOVASCULAR CENTER, Osaka (JP)

(72) Inventors: Yasuyuki KATAOKA, Sunnyvale, CA (US); Yukiko FUKUDA, Sunnyvale, CA (US); Jon PETERSON, Sunyvale, CA (US); Kazunori UEMURA, Osaka (JP); Kenji SUNAGAWA, Osaka (JP)

(73) Assignees: NTT RESEARCH, INC., Sunnyvale, CA (US); NATIONAL CEREBRAL AND CARDIOVASCULAR CENTER, Osaka (JP)

(21) Appl. No.: 19/166,329

(22) PCT Filed: Mar. 29, 2024
(86) PCT No.: PCT/US2024/022360

§ 371 Date: Sep. 17, 2025

Related U.S. Application Data
(60) Provisional application No. 63/493,609, filed on Mar. 31, 2023.

(51) Int. Cl. G16H 70/40

(52) U.S. Cl. CPC G16H 70/40

(57) ABSTRACT

Embodiments disclosed herein establish the relationship between changes in cardiovascular metrics and a combination of drugs. The relationship is established based on tracing a chain of causality from drug combination to cardiovascular parameters, described by a linear relationship, and from cardiovascular parameters to cardiovascular metrics, by measuring the direction and sensitivity of the hemodynamic changes caused by the changes to the cardiovascular parameters (which in turn is caused by administration of drugs). Some embodiments model myocardial oxygen consumption—which cannot be measured directly—and also establish the relationship between hemodynamic changes to the myocardial oxygen consumption and change in one or more cardiovascular parameters. These embodiments assist in clinical decision making because the chain of causality from the drugs to the cardiovascular metrics is both qualified and quantified; thereby reducing the level of guesswork and unknowns in deciding an optimal combination of drugs for acute heart failure patients.

### CROSS REFERENCE TO RELATED APPLICATIONS

This disclosure is related to and claim priority from U.S. Provisional Application No. 63/493,609 filed Mar. 31, 2023, and entitled “Assisting Clinical Decision-Making in Drug Therapy for Acute Heart Failure Patients,” which has been incorporated by reference in its entirety.

This disclosure is related to U.S. Provisional Application No. 63/346,143, filed May 26, 2022, and entitled “Optimizing Drug Combinations for Treating Acute Heart Failure,” which has been incorporated by reference in its entirety.

### FIELD

This disclosure relates to systems, methods, and devices for assisting clinical decision making for determining an optimal combination of drugs to reach target cardiovascular metrics in acute heart failure patients.

### BACKGROUND

Acute heart failure is caused by different factors and therefore generally requires complex drug therapies with multiple drugs (also referred to as medications). Some examples of the drugs for treating acute heart failure include positive inotropes such as dobutamine, vasopressors such as norepinephrine, vasodilators such as sodium nitroprusside, fluids such as dextran, diuretics such as furosemide, etc. Each of these drugs may treat a different, specialized aspect of acute heart failure. For a more effective treatment of acute heart failure, a combination of these drugs is generally required.

Furthermore, acute heart failure has detrimental effects on multiple organs of the body, including the heart. Particularly, acute heart failure changes cardiovascular metrics of the heart, and these changes cause the detrimental effects on the other organs. In a clinical setting, therefore, it is desirable to stably maintain the cardiovascular metrics within safe boundaries such that problems arising from acute hear failure do not cascade over to other organs.

The technical challenge, however, is the complexity and lots of unknowns. As described above, there are different drugs, each impacting one aspect of the heart and producing its specific side effects. For example, even when just two drugs are administered, the effects—both positive and negative—are unknown and difficult to predict. Clinical decision making is therefore driven by trial and error and guesswork; and is inherently inaccurate. Such undesirable situation creates unnecessary hardships on the patients. The health outcomes are less than desirable and mortality rate among heart failure patients remains unnecessarily high.

As such, a significant improvement in systems, methods, and devices to assist clinical decision-making for an optimal combination of drugs to achieve a desired level of cardiovascular metrics in acute heart failure patients is therefore desired.

### SUMMARY

In some embodiments, a system may be provided. The system may include a non-transitory storage medium storing computer program instructions and one or more processors configured to execute the computer program instructions to cause operations. The operations may include receiving a plurality of current cardiovascular metrics for a patient with acute heart failure and executing a clinical decision making assistance module on the plurality of current cardiovascular metrics to generate a recommended combination of drugs to reach a plurality of target cardiovascular metrics. The clinical decision making assistance module may be based on a drug library linearly modeling a pathway from a plurality of current cardiovascular parameters associated with the plurality of current cardiovascular metrics to a plurality of target cardiovascular parameters associated with the plurality of target cardiovascular metrics and a mapping between the plurality of target cardiovascular metrics and the plurality of target cardiovascular parameters, the mapping including a time transient directional response of one or more cardiovascular metrics due to a change in one or more cardiovascular parameters caused by an administration of one or more drugs. The operations may further include outputting the recommended combination of drugs.

In some embodiments, a computer-implemented method may be provided. The method may include receiving, from a client device by a computing system, a plurality of current cardiovascular metrics for a patient with acute heart failure and executing, by the computing system, a clinical decision making assistance module on the plurality of current cardiovascular metrics to generate a recommended combination of drugs to reach a plurality of target cardiovascular metrics. The clinical decision making assistance module may be based on a drug library linearly modeling a pathway from a plurality of current cardiovascular parameters associated with the plurality of current cardiovascular metrics to a plurality of target cardiovascular parameters associated with the plurality of target cardiovascular metrics and a mapping between the plurality of target cardiovascular metrics and the plurality of target cardiovascular parameters, the mapping including a time transient directional response of one or more cardiovascular metrics due to a change in one or more cardiovascular parameters caused by an administration of one or more drugs. The method may further include outputting, by the computing system to the client device, the recommended combination of drugs.

In some embodiments, non-transitory storage medium storing computer program instructions is provided. The computer program instructions, which when executed by one or more processors may cause operations, which may include receiving a plurality of current cardiovascular metrics for a patient with acute heart failure and executing a clinical decision making assistance module on the plurality of current cardiovascular metrics to generate a recommended combination of drugs to reach a plurality of target cardiovascular metrics. The clinical decision making assistance module may be based on a drug library linearly modeling a pathway from a plurality of current cardiovascular parameters associated with the plurality of current cardiovascular metrics to a plurality of target cardiovascular parameters associated with the plurality of target cardiovascular metrics and a mapping between the plurality of target cardiovascular metrics and the plurality of target cardiovascular parameters, the mapping including a time transient directional response of one or more cardiovascular metrics due to a change in one or more cardiovascular parameters caused by an administration of one or more drugs. The operations may further include outputting the recommended combination of drugs.

### BRIEF DESCRIPTION OF DRAWINGS

FIG. 1 depicts an example computing environment for assisting clinical decision-making in drug therapy for acute heart failure patients, according to example embodiments of this disclosure.

FIG. 2 depicts an example analytical model, according to example embodiments of this disclosure.

FIG. 3 depicts a flow diagram of an example method, based on the example embodiments of this disclosure.

FIG. 4 depicts example charts visualizing the hemodynamic directions of the real-world drug and the predicted response based on the analytical model, according to example embodiments of this disclosure.

FIGS. 5A-5E depict example charts visualizing a single drug infusion simulation, based on the example embodiments of this disclosure.

FIG. 6 depicts example charts visualizing three acute heart failure scenarios simulation, based on example embodiments of this disclosure.

FIG. 7 depicts an example chart visualizing simulation using Forrester classification, according to example embodiments of this disclosure.

FIG. 8 shows a block diagram of an example computing device that implements various features and processes, according to example embodiments of this disclosure.

### DESCRIPTION

It is desirable to keep cardiovascular metrics (e.g., left atrial pressure, cardiac output, mean arterial pressure, or myocardial oxygen consumption) within a stable range for acute heart failure patients. There may be detrimental effect on other organs, and the heart itself, if the cardiovascular metrics are outside of the range. A combination of drugs (e.g., positive inotropes, vasopressors, vasodilators, fluids, or diuretics) is used to induce hemodynamic changes to the cardiovascular metrics. The hemodynamic changes induced by the drugs can be modeled as the drugs changing cardiovascular parameters (systemic vascular resistance, cardiac contractility, heart rate, or stressed blood volume), which in turn change the cardiovascular metrics.

Embodiments disclosed herein assist clinical decision making on the combination drugs to be administered to reach a target range for the cardiovascular metrics. For example, some embodiments establish the relationship between changes in cardiovascular metrics (also referred to as hemodynamic changes) and the combination of drugs. The relationship is established based on tracing a chain of causality from drug combination to cardiovascular parameters, described by a linear relationship, and from cardiovascular parameters to cardiovascular metrics, by measuring the direction and sensitivity of the hemodynamic changes caused by the changes to the cardiovascular parameters (which in turn is caused by administration of drugs). Some embodiments model myocardial oxygen consumption-which cannot be measured directly- and also establish the relationship between hemodynamic changes to the myocardial oxygen consumption and change in one or more cardiovascular parameters. These embodiments assist in clinical decision making because the chain of causality from the drugs to the cardiovascular metrics is both qualified and quantified; thereby reducing the level of guesswork and unknowns in deciding an optimal combination of drugs for acute heart failure patients.

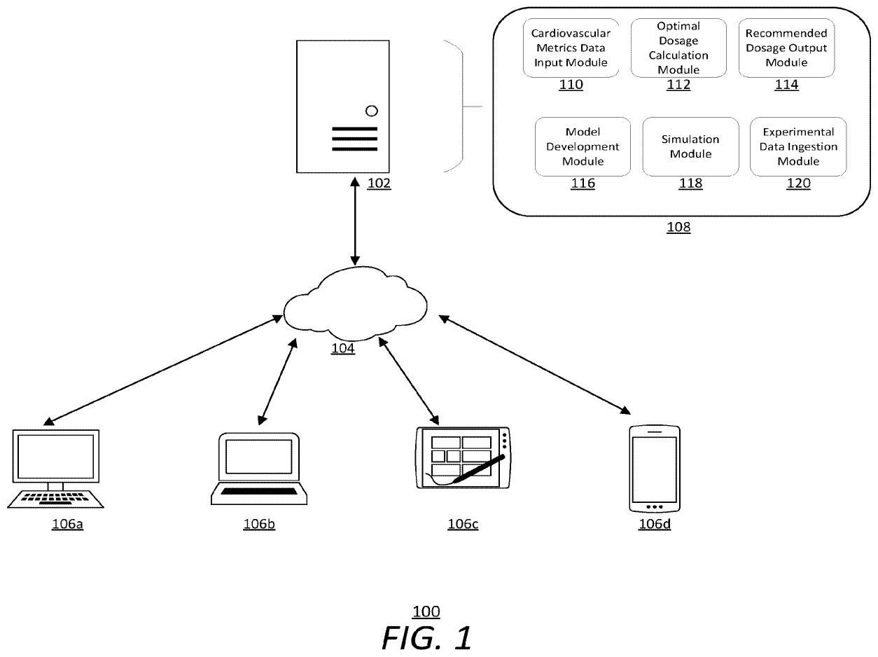

FIG. 1 depicts an example computing environment 100 for assisting clinical decision-making in drug therapy for acute heart failure patients, according to example embodiments of this disclosure. As shown, the computing environment 100 may be based on a client-server model, with a server 102 connected to multiple clients 106a-106d (commonly referred to as a client 106 or collectively referred to as clients 106) via a network 104. It should, however, be understood that the client-server model is just for illustration and ease of explanation and should not be considered limiting. Therefore, any type of computing environment performing the functionality disclosed herein should be considered within the scope of this disclosure. Furthermore, the individual components of the computing environment 100 are just illustrative and computing environments with alternative, additional, or fewer number of components should be considered within the scope of this disclosure.

The computing environment 100 may be generally in a clinical setting to assess clinical decision making for acute heart failure patients. In some example use cases, the server 102 may store different software modules 108 that may be accessed by the clients 106 using the network 104. The clients 106 themselves may have standalone applications (not shown) to access the software modules 108. Alternatively, the clients 106 may access the software modules through a browser application, for example.

The hardware of the server 102 storing the software modules 108 may include any kind of computing device. For example, the server 102 may include any kind of computing device, including but not limited to a server computer, a desktop computer, a laptop computer, a tablet computer, a smartphone. The server 102 may not necessarily be at a single location and may be realized by a network of computers. Furthermore, the server 102 may not necessarily be co-located within the clinical setting itself, and may be hosted by a third party cloud computing provider. Therefore, any kind of server 102 should be considered within the scope of this disclosure.

As described above, the clients 106 may access the server 102 through the network 104. The network 104 may include any combination of one or more packet switching networks (e.g., an IP based network) and one or more circuit switching networks (e.g., a cellular telephony network). Some non-limiting examples of the network 104 include a local area network, a metropolitan area network, a wide area network such as the Internet, etc. Similarly, non-limiting examples of the clients 106 may include a desktop terminal (e.g., desktop terminal 106a), a laptop computer (e.g., a laptop computer 106b), a tablet computer (e.g., a tablet computer 106c), a smartphone (e.g., a smartphone 106d), etc. Any type of computing device that allows an access to the server 102 through the network 104 should be considered within the scope of this disclosure. Furthermore, the functionality described within this disclosure can be distributed in any fashion, i.e., functionality of the server 102 may be performed by one or more clients 106 and vice versa.

As described above, the server 102 may include multiple software modules. FIG. 1 shows some non-limiting example software modules: a cardiovascular metrics data input module 110, an optimal dosage calculation module 112, a recommended dosage output module 114, a model development module 116, a simulation module 118, and an experimental data ingestion module 120. It should be understood that this described modularization of the server 102 functionality is just for the ease of explanation and should not be considered limiting. Therefore, any kind of alternative modularization should be considered within the scope of this disclosure.

The cardiovascular metrics data input module 110 may receive cardiovascular metrics data from the clients 106. The received cardiovascular metric data may include current cardiovascular metrics for a patient and target cardiovascular metrics. For instance, as the current cardiovascular metrics, the cardiovascular metrics data input module 110 may receive one or more of the current measurements of left atrial pressure, cardiac output, mean arterial pressure, or myocardial oxygen consumption. Similarly, as the target cardiovascular metrics, the cardiovascular metric data input module may receive one or more of the desired measurements of left atrial pressure, cardiac output, mean arterial pressure, or myocardial oxygen consumption. The cardiovascular metrics data input module 110 may receive the cardiovascular metrics data (current and/or target) from one or more of the clients 106 and through the network 104. In some embodiments, the cardiovascular metrics data input module 110 may support real-time data input, where the server 102 processes the received cardiovascular metrics data in real-time. In other embodiments, the cardiovascular metrics data input module 110 may support batch processing, where individual cardiovascular metrics data are batched (e.g., buffered or stored), and the processing may be performed together for the batched data (e.g., during off-peak hours for the server 102). Therefore, the cardiovascular metrics data input module 110 may manage the receipt of data from the clients 106 for any type of processing.

The optimal dosage calculation module 112 may calculate optimal dosage for the received cardiovascular metrics data. For example, the optimal dosage calculation module 112 may use the received current cardiovascular metrics data to compare it against a target and/or received desired cardiovascular metrics, and invoke one or more analytical models disclosed herein to calculate the optimal dosage of drugs. The optimal dosage may be based on, for example, both the direction and sensitivity of changes to the cardiovascular metrics caused by the drugs. (It should be understood that the embodiments disclosed herein model this direction and sensitivity of the changes as the drugs changing measurable cardiovascular parameters, which in turn change the cardiovascular metrics). The optimal dosage calculation module 112 may, therefore, consider the different effects—a first drug may cause a change in one direction for a cardiovascular metric and a second drug may cause a change in an opposite direction—to generate a recommended combination. Generally, the optimal dosage calculation module 112 may utilize one or more analytical models on the specific cardiovascular metrics received by the cardiovascular metrics data input module 110 to calculate the optimal dosage.

The recommended dosage output module 114 may provide a recommended dosage to a clinician. Specifically, the recommended dosage output module 114 may take in the optimal dosage calculated by the optimal dosage calculation module 112, format the calculated optimal dosage to a target format, and provide it to the clinician. For example, if the clinician provided cardiovascular metrics data using the desktop terminal 106a soliciting advice on dosage, the recommended dosage output module 114 may transmit back the recommended dosage (i.e., the optimal dosage calculated by the optimal dosage calculation module 112) to the desktop terminal 106a. In some embodiments, however, the clinician may provide the cardiovascular metrics data using the desktop terminal 106a but may seek to receive the recommended dosage on the smartphone 106d. In this situation, the recommended dosage output module 114 may format the recommended dosage to the format compatible with the smartphone 106d and provide the formatted recommended dosage to the smartphone 106d. Furthermore, the recommended dosage can be provided in any format, e.g., displayed on an application window, displayed on a browser window, an e-mail, a text message, and/or any other format.

While the software modules 110, 112, and 114 generally interface the clinicians; software modules 116, 118, and 120 may generally interface the model developers. That is, model developers may use one or more of the clients 106 to access these software modules to develop, modify, and/or refine the analytical models described throughout this disclosure.

The model development module 116 may allow a model developer to develop one or more analytical modules. For instance, the model development module 116 may provide an interface, e.g., a graphical user interface, for the model developer to define one or more analytical models. Furthermore, the model development module 116 may allow the model developer to upload and/or port a pre-defined analytical module to the server 102. The model development module 116 may therefore generally provide any kind of computing environment support to develop the analytical models described throughout this disclosure.

The simulation module 118 may allow the model developer to simulate the analytical models (e.g., defined/developed/ported using the model development module 116). The simulations may include, for example, numerical simulation, where collected numerical data may be used on the analytical models to observe the outputs. The simulations may produce, for example, numerical data, graphical data, and/or any other type of output data. The simulation module 118 may generally allow for validations of the analytical models based on these simulations (e.g., to determine whether the analytical models perform as desired with simulated scenarios).

The experimental data ingestion module 120 may ingest experimental data used for model development. For instance, the experimental data may include measured cardiovascular parameters and/or metrics of animals. Such experimental data may be used by the simulation module 118 to simulate the analytical models. Other experimental data may include a detailed experimental data containing both inputs and outputs, and can be used to compare the real-world results with the simulated results. In yet another example, the experimental data may include a continuous stream of data as the patients are being treated in clinical settings, which may be used to continuously modify and/or refine the analytical models. Therefore, the experimental data ingestion module 120 may receive any kind of real-world numerical data that may be used to develop, refine, and/or modify the analytical modules disclosed throughout this disclosure.

After one or more analytical models have been developed and validated, clinicians may use the software modules 108 within the server 102 to aid their clinical decision-making process for acute heart failure patients. In an example operation, a clinician uses an interface in a client 106 to enter current cardiovascular metrics such as the four-dimensional metrics (1. left atrial pressure, 2. cardiac output, 3. mean arterial pressure, and 4. myocardial oxygen consumption) described throughout this disclosure. Alternatively or additionally, the clinician may enter the current cardiovascular parameters. The client 106 may then transmit the entered metrics (and/or parameters) to the server 102 through the network 104. The server 102 may deploy the software modules 108 to calculate a recommended dosage for the current cardiovascular metrics to a target cardiovascular metrics (in some embodiments, the clinician may provide target cardiovascular metrics along with the current cardiovascular metrics). Particularly, the cardiovascular metrics data input module 110 may receive the input cardiovascular metrics (current and/or target) and process the input. Then, the optimal dosage calculation module 112 may deploy one or more analytical models on the input cardiovascular metrics to generate an optimal dosage. The recommended dosage output module 114 may then transmit the calculated optimal dosage as recommended dosage back to the requesting clinician. One or more of the model development module 116, simulation module 118, or experimental data ingestion module 120 may use the above described cycle and feedback, if any, to refine one or more of the analytical models.

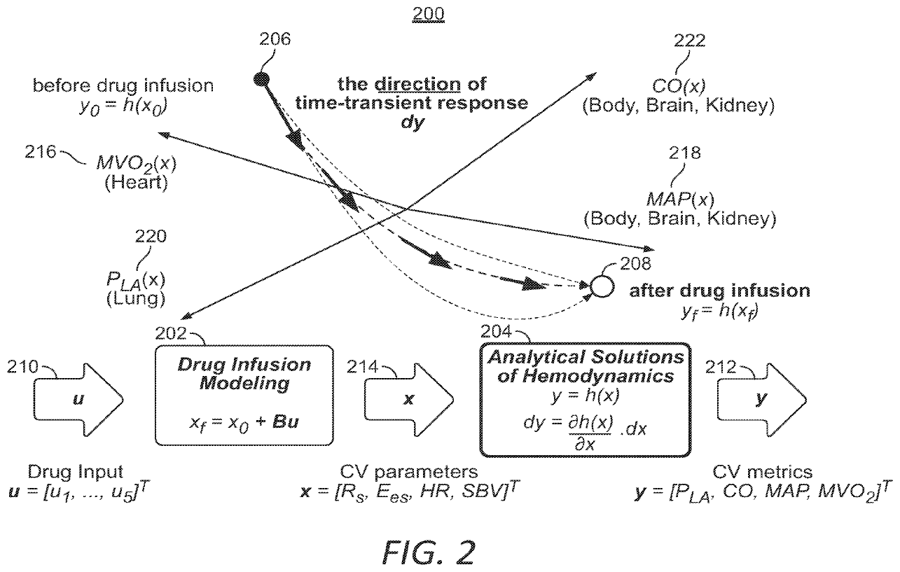

FIG. 2 depicts an example analytical model 200, according to example embodiments of this disclosure. The example analytical model 200 may be used by the software modules 108 described in FIG. 1. For instance, the model development module 116 may be used to define and/or port the analytical model 200, the simulation module 118 may be used to perform a numerical simulation of the analytical model 200, and the experimental data ingestion module 120 may be used to receive experimental data to validate, modify, and/or refine the analytical model. Furthermore, the cardiovascular metrics data input module 110 may receive cardiovascular metrics data to be used by the analytical model 200, the optimal dosage calculation module 112 may use the analytical model 200 to calculate an optimal dosage, and the recommended dosage output module 114 may output the optimal dosage calculated by the analytical model 200 to a format compatible with a target device/platform. It should further be understood that the analytical model 200 is just an example and should not be considered limiting: analytical models with additional, alternative, or fewer number of steps, and/or components should be considered within the scope of this disclosure. As shown, the example analytical model 200 divides the chain of causality from a drug input 210 to cardiovascular metrics 212 as a linear drug infusion model 202, to generate cardiovascular parameters 214 from the drug input 210, and a hemodynamics model 204 to generate cardiovascular metrics 212 from the cardiovascular parameters 214. To put it differently, the drug infusion model 202 and the hemodynamics model 204 incorporate the changes in the cardiovascular parameters 214 before drug infusion, y₀−h(x₀), labeled as initial state 206 to the cardiovascular parameters 214 after the drug infusion y_{f}=h(x_{f}), labeled as a final state 208. Considering all the complexities described throughout the disclosure, the goal is to find the optimal drug input 210 (a combination of different drugs) that generates a desired (also referred to as target) final state 208 corresponding to desired (also referred to as target) cardiovascular metrics 212.

The drug input 210 may include a combination of different drugs, defined by the vector u=[u₁, u₂, u₃, u₄, u₅]^{T}ϵ [symbol] ⁵, i.e., a vector in a 5-dimensional space. Each element of the vector u may correspond to a drug that is used to treat acute heart failure. Example drugs may include Dobutamine as a positive inotrope, Norepinephrine as a vasopressor, Sodium Nitroprusside as a vasodilator, Dextran as a fluid, Furosemide as a diuretic, etc. As a generalized example, u₁ may correspond to positive inotropes, u₂ may correspond to vasopressors, u₃ may correspond to vasodilators, u₄ may correspond to fluids, and u₅ may correspond to diuretics. It should however be understood that these are just example drugs forming an example combination and should not be considered limiting. Therefore, a generalized input vector u=[u₁, . . . uₙ]^{T} should be considered within the scope of this disclosure.

The cardiovascular parameters 214 may include, for example, systemic vascular resistance Rₛ (measured in mmHg sec/ml), cardiac contractility Eₑₛ (measured in mmHg/ml), heart rate HR (measured in beats/min), and stressed blood volume SBV (measured in ml). Systemic vascular resistance may be defined as a resistance in the vascular system that is used to create a blood pressure, e.g., blood vessels may constrict to increase Rₛ. Cardiac contractility may indicate an innate ability of the heart muscle to contract. Heart rate may indicate the number of heart beats per unit time, e.g., beats per minute. Stressed blood volume may be defined as any volume of blood above a baseline volume (i.e., unstressed) of blood required to fill blood vessels to exert pressure thereon. In the shown analytical model 200, the cardiovascular parameters 214 may be defined as x=[Rₛ,Eₑₛ,HR,SBV]^{T}ϵ [symbol] . As further shown in the analytical model 200, x₀ corresponds to the initial state 206 (of the cardiovascular parameters 214) and x_{f} corresponds to the final state 208 (of the cardiovascular parameters 214). It should be understood that these cardiovascular parameters 214 are merely examples, and other parameters should be considered within the scope of this disclosure. Therefore, as with the vector u, vector y may also be generalizable to a vector having n elements, not just the shown four elements.

The cardiovascular metrics 212 may include, for example, left atrial pressure P_{LA} (measured in mmHg), cardiac output CO (measured in L/min), mean arterial pressure MAP (measured in mmHg), myocardial oxygen consumption MVO₂ (measured in ml O₂/min/100 g), etc. Left atrial pressure is generally pressure generated during the contraction of the left atrium. Cardiac output is generally the volume of blood ejected out of the heart per unit time, which can be represented as a product of heart rate and stroke volume. Mean arterial pressure is generally defined as average arterial pressure throughout one cardiac cycle, including both systole and diastole. Myocardial oxygen consumption generally approximates the oxygen used by the heart, principally for cardiovascular contraction. The cardiovascular metrics may be presented as a vector y=[P_{LA},CO,MAP,MVO₂]^{T}ϵ [symbol] . It should be understood that these cardiovascular metrics 212 are merely examples, and other metrices should be considered within the scope of this disclosure. Therefore, as with the vector u and x, vector y may also be generalizable to a vector having n elements, not just the shown four elements.

As discussed above, the chain of causality goes from the drug input 210 to the cardiovascular parameters 214 and then to the cardiovascular metrics 212. For the first part of the chain, the cardiovascular parameters 214 can therefore be represented by a vector function of the drug input, i.e., x=ƒ(u)ϵ [symbol] . The function ƒ(u) in a steady state can be modeled as a linear function, x_{ƒ}=Bu+x₀, where x₀ indicates initial cardiovascular parameters 214 prior to the drug input 210, x_{f} indicates final cardiovascular parameters 214 after the drug input 210, and input matrix B indicates drug library that represents the multi-dependency effect of each drug to the cardiovascular parameters 214 in the steady state.

In the second part of the chain of causality, the cardiovascular parameters 214 (i.e., the changes thereto) will modulate the cardiovascular metrics 212 (or cause hemodynamic changes to the cardiovascular metrics 212). The analytical solution of P_{LA}, CO, MAP can be derived using an intersection of Frank-Starling Curve (as known in the art) and Guyton's Venous Return Curve formula (as known in the art), as follows:

$${P}_{L A} ( x ) = - α ( \frac{a W ( - \frac{b}{a} exp ( \frac{c}{a} ) )}{b} ) - α [ mmHg ]$$

$$CO ( x ) = - aW ( - \frac{b}{a} exp ( \frac{c}{a} ) ) + c [ \frac{L}{min} ]$$

$$MAP ( x ) = \frac{1000}{60} CO ( x ) {x}_{1} [ mmHg ]$$

$$a = \frac{1 {x}_{2} {x}_{3}}{100 {0}_{β ( {x}_{2} + \frac{{x}_{3}}{60} {x}_{1} )}}$$

$$b = \frac{60 α}{1000} \frac{α}{{R}_{vp}}$$

$$c = \frac{60}{1 \; 000} \frac{11}{{R}_{vp}} ( \frac{{x}_{4}}{{C}_{s} + {C}_{p}} + α )$$

where W(⋅) is defined as the Lambert function (as known in the art); where α and β are the end-diastolic pressure-volume relationship (ED-PVR) parameters; and where Rᵥₚ, Cₚ, Cₛ are, respectively, the resistance for pulmonary venous return, the compliance in the pulmonary circulation, and the systemic circulation.

The remaining cardiovascular metric 212 to be analytically derived is MVO₂. It is known that metabolic demand of the heart itself—as indicated by the MVO₂ measurement—is a key indicator to prevent poor prognosis, re-hospitalization, adverse cardiovascular events, and/or mortality for acute heart failure patients. Embodiments disclosed herein use the mechanical energy generated by ventricular contraction, e.g., left ventricular contraction, to analytically derive MVO₂, e.g., for the left ventricle as a function of other cardiovascular metrics 212, by using the following function:

$${MV O}_{2} ( x ) = ( {A}_{o} PV A ( x ) + {B}_{o} {x}_{2} + {C}_{o} ) {x}_{3}$$

where A₀, B₀, and C₀ are constant parameters shown in Table I below, and where PVA(x) (measured in mmHg·ml/100 g/beat) is the normalized pressure volume area for 100 g of the left ventricle.

TABLE I
Constant Parameters of MVO2 model

| Param. | Value | Unit |
|---|---|---|
| Aₒ | 1.8 × 10⁻⁵ | [ml O₂/mmHg/ml] |
| Bₒ | 2.4 × 10⁻³ | [ml O₂ ml/mmHg/beat/100 g] |
| Cₒ | 1.4 × 10⁻² | [ml O₂/beat/100 g] |

With the assumption that the weight of the left ventricle is about 0.4% of the total body weight (BW), a normalized PVA(x) can be represented as cardiovascular metrics 212 by:

$$PVA ( x ) = ( \frac{{MAP ( x )}^{2}}{2 {x}_{2}} + \frac{CO ( x ) MAP ( x )}{{x}_{3}} ) \frac{25}{BW}$$

Therefore, MVO₂ can also be represented as a function of other cardiovascular metrics 212 (e.g., CO 222 and MAP 218).

While the final state 208 is reached from the initial state 206 traversing the overall pathway, the response direction and sensitivity to the drug input 210 may be different for each of the cardiovascular metrics. For example, the direction of response of MVO₂ 216 (generally affecting the heart) may be opposite of MAP 218 (generally affecting body, brain, and kidney), and the direction of response of P_{LA} 220 (generally affecting lungs) may be opposite of CO 222 (generally affecting body, brain, kidney). That is, for example, the drug input 210 may increase CO 222 while decreasing P_{LA} 220. Embodiments disclosed herein use the analytical model 200 to analyze the different directions—and sensitivities thereto—of the changes of the cardiovascular metrics 212 (i.e., the hemodynamic changes).

To determine the direction of a hemodynamic change, it follows from fundamental calculus that these changes can be represented as quantification of a slight change in the cardiovascular metrics 212, dy, as a result of a slight change in the cardiovascular parameters 214, dx. Let the cardiovascular metrics 212 (y) be represented by a vector function.

$$h ( x ) := {[ {P}_{LA} ( x ) , CO ( x ) , MAP ( x ) , {MVO}_{2} ( x ) ]}^{T} ∈ {ℝ}^{4}$$

Then, the direction of the hemodynamic change may be derived by the total derivative of y, represented as

$$dy = \frac{\partial h ( x )}{\partial x} dx = [ \begin{matrix}〈 ∇ CO ( x ) , dx 〉 \\ 〈 ∇ {P}_{LA} ( x ) , dx 〉 \\ 〈 ∇ MAP ( x ) , dx 〉 \\ 〈 ∇ {MVO}_{2} ( x ) , dx 〉\end{matrix} ]$$

$$\frac{\partial h ( x )}{\partial x} = [ \begin{matrix}\frac{\partial CO ( x )}{\partial {R}_{s}} & \frac{\partial CO ( x )}{\partial {E}_{es}} & \frac{\partial CO ( x )}{\partial HR} & \frac{\partial CO ( x )}{\partial SBV} \\ \frac{\partial {P}_{LA} ( x )}{\partial {R}_{s}} & \frac{\partial {P}_{LA} ( x )}{\partial {E}_{es}} & \frac{\partial {P}_{LA} ( x )}{\partial HR} & \frac{\partial {P}_{LA} ( x )}{\partial SBV} \\ \frac{\partial MAP ( x )}{\partial {R}_{s}} & \frac{\partial MAP ( x )}{\partial {E}_{es}} & \frac{\partial MAP ( x )}{\partial HR} & \frac{\partial MAP ( x )}{\partial SBV} \\ \frac{\partial {MVO}_{2} ( x )}{\partial {R}_{s}} & \frac{\partial {MVO}_{2} ( x )}{\partial {E}_{es}} & \frac{\partial {MVO}_{2} ( x )}{\partial HR} & \frac{\partial {MVO}_{2} ( x )}{\partial SBV}\end{matrix} ]$$

$$dx = {[ {dR}_{s} , {dE}_{es} , dHR , dSBV ]}^{T} .$$

It follows from above that in the Jacobian matrix above (representing

$$\frac{\partial h ( x )}{\partial x} ) ,$$

the columns show the sensitivity of how a change in a single cardiovascular parameter 214 (xᵢ) affects multiple cardiovascular metrics 212. On the other hand, a row of the Jacobian matrix, which can also be expressed as

$$∇ {h}_{i} ( x ) = [ \frac{\partial {h}_{i} ( x )}{\partial {R}_{s}} , \frac{\partial {h}_{i} ( x )}{\partial {E}_{es}} , \frac{\partial {h}_{i} ( x )}{\partial HR} , \frac{\partial {h}_{i} ( x )}{\partial SBV} ]$$

shows the sensitivity of how the changes in multiple cardiovascular parameters 214 affect a single cardiovascular metric 212 (yᵢ). The above Jacobian matrix may be analytically solved using the derivate of the Lambert function W, described above, as follows:

$$\frac{dW ( z )}{dz} = \frac{1}{z + {e}^{W ( z )}}$$

where

$$( z \neq - \frac{1}{e} )$$

The above analytical derivations show that the cardiovascular metrics 212 can be expressed in the form of the cardiovascular parameters 214, and the hemodynamics of the cardiac metrics (i.e., dy) can be expressed in the form of the change in the cardiac parameters. The cardiovascular parameters 214 in turn can be controlled by the drug input based on the drug infusion model 202. For the drug infusion model, the drug library B can be determined in one or more embodiments as described below.

For example, the drug library B can be determined using animal experimentation. In one experiment, a dog was anesthetized, and the bilateral carotid baroreceptors and vagal trunk were denervated. A thoracotomy was performed, after which the dog was connected to a system that measures MAP from the right femoral artery, CO via ultrasonic flow meter around the ascending aorta, both P_{LA} and right atrial pressure (P_{RA}) directly in the corresponding atrium, and HR. Prior to drug infusion, a baseline was measured for one minute. Next, a single drug was administered for 10 minutes to measure pharmacological effect until steady state. Then, minimal washout time (i.e., drug being cleared from the dog's system) was ensured until MAP stabilized at the pre-drug value. These procedures were repeated for other drugs. The dosages for the drugs administrated in this animal experiment were Dobutamine: 5.0 μg/kg/min, Norepinephrine: 0.15 μg/kg/min, Sodium Nitroprusside 5.0 μg/kg/min, and Dextran: 5.0 ml/kg. It should further be noted this animal-based experimentation protocol was approved by the animal subjects committee of the National Cerebral and Cardiovascular Center (NCVC), Japan.

Using the above experimentation, the drug library B was developed. The gains from each drug input uᵢ (each element of the drug input 210) to each cardiovascular parameter xᵢ (each element of the cardiovascular parameters 214) change were identified by fitting to a first order single-input single-output process model with dead time. In this setup Rₛ is computed by 60 (MAP-P_{RA})/CO, Eₑₛ is estimated by an estimation method based on cardiac mechanics, HR is measurable from a sensor, SBV is stressed blood volume estimated by circulatory equilibrium framework. Aggregating these identified gains, the drug library B was found to be (noting that Furosemide is assumed to decrease only SBV):

$$B = [ \begin{matrix}- 0.0335 & 3.33 & - 0.419 & - 0.00725 & 0 \\ 3.88 & 26.1 & - 1.14 & - 0.0135 & 0 \\ 8.10 & 24.9 & 0.164 & - 0.450 & 0 \\ 27.7 & 76.2 & - 14.1 & 1.47 & - c \cdot BW\end{matrix} ] .$$

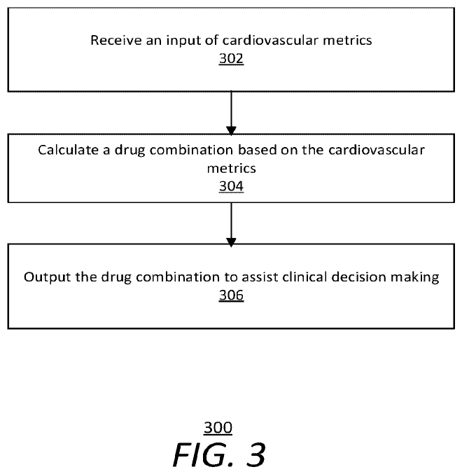

FIG. 3 depicts a flow diagram of an example method 300, based on the example embodiments of this disclosure. The example method 300 may be performed by any combination of components of the computing environment 100 shown in FIG. 1, using any portion of the analytical model 200 shown in FIG. 2. It should be understood that the steps of the method 300 are just examples and should not be considered limiting. Methods with additional, alternative, or fewer number of steps should be considered within the scope of this disclosure.

The method 300 may begin at step 302. At step 302, server 102 may receive an input of cardiovascular metrics. It should be understood that the clinician may enter current cardiovascular parameters in lieu of or in addition to the current cardiovascular metrics. For example, a desktop terminal in a hospital terminal may be used by a clinician to enter the cardiovascular metrics and/or the cardiovascular parameters. Alternatively, the clinician may enter the cardiovascular metrics and/or the cardiovascular parameters on a smartphone or a tablet computer. The cardiovascular metrics may include, for example, current cardiovascular metrics and/or target cardiovascular metrics.

At step 304, server 102 may calculate a drug combination based on the entered cardiovascular metrics and/or parameters. In some embodiments, regardless of the modality of the entry of the desired cardiovascular metrics and/or parameters, step 304 may be executed to calculate a drug combination for the target cardiovascular metrics. The drug combination may be calculated based on the analytical models described throughout this disclosure.

At step 306, server 102 may output the drug combination (e.g., at the requesting device) to assist clinical decision making. That is, the clinician can rely on the tested and simulated models to aid the decision making and rely less on guesswork.

As described above, one or more analytical models may be validated using animal-based experiments. One of the purposes of the animal-based experiments is to validate the direction of hemodynamic change dy, as shown by the equations above. The proposed direction (i.e., direction derived analytically) was compared to the real-world direction of hemodynamic change in animal experiments, such as the one described above. The performance (real-world vs. analytical) was evaluated in (P_{LA}, CI) space because this is a common metrics known as a Forrester classification in acute heart failure treatment, where CI is cardiac index defined by CO/BSA (body surface area).

For this comparative analysis, the real-word responses from the animal experiment after the drug infusion are donated by P_{LA}, and CIᵣ. To evaluate the direction (e.g., using angle), P_{LA_r} and CIᵣ are to be normalized because they have different dimensions and data ranges. Given the time step k that increments every 30 seconds and measured cardiovascular parameters x[k], let the normalized vector of real response at the time of k be:

$${dy}_{r} [ k ] = ( \frac{{P}_{LA \_ r} [ k + 1 ] - {P}_{LA \_ r} [ k ]}{\overline{{P}_{LA \_ r}}} , \frac{{CI}_{r} [ k + 1 ] - {CI}_{r} [ k ]}{\overline{{CI}_{r}}} )$$

where the denominators P̅_̅{̅L̅A̅}̅_̅{̅ᵣ̅}̅ and C̅I̅ᵣ̅ are the peak-to-peak values in each dimension.

In some embodiments, the proposed directions of the hemodynamic change are considered in two different versions. The first version may be the original total derivative that uses the one-step future cardiovascular parameters x[k+1], as follows:

$${dy}_{f} [ k ] = ( {dP}_{LA \_ f} [ k ] , {dCI}_{f} [ k ] )$$

$${dP}_{LA \_ f} [ k ] = \frac{〈 ∇ {P}_{LA} ( x [ k ] ) , x [ k + 1 ] - x [ k ] 〉}{\overline{{P}_{LA \_ r}}}$$

$${dCI}_{f} [ k ] = \frac{〈 ∇ CI ( x [ k ] ) , x [ k + 1 ] - x [ k ] 〉}{\overline{{CI}_{r}}} .$$

The second version may be an approximated total derivative that uses the one-step past cardiovascular parameters x[k−1], as follows:

$${dy}_{p} [ k ] = ( {dP}_{LA \_ p} [ k ] , {dCI}_{p} [ k ] )$$

$${dP}_{LA \_ p} [ k ] = \frac{〈 ∇ {P}_{LA} ( x [ k ] ) , x [ k ] - x [ k - 1 ] 〉}{\overline{{P}_{LA \_ r}}}$$

$${dCI}_{p} [ k ] = \frac{〈 ∇ CI ( x [ k ] ) , x [ k ] - x [ k - 1 ] 〉}{\overline{{CI}_{r}}} .$$

While dyⱼ[k] generated by using of one-step future cardiovascular parameter may be more accurate, the use of dyₚ[k] (i.e., one step past) may be more practical for an example application that predicts the hemodynamic direction based on past information. Table II below shows constant parameters that were tuned by body weight and used in the animal experiments.

TABLE II
Constant Parameters used in Experiments

| Param. | Value | Unit (γ = μg/kg/min) | Description |
|---|---|---|---|
| α | 0.86 | unitless | EDPVR |
| β | 0.15 | unitless | EDPVR |
| Cₚ | 3.0 | [ml/mmHg] | Pulmonary Compliance |
| Cₛ | 17 | [ml/mmHg] | Systemic Compliance |
| Rᵥₚ | 0.25 | [mmHg · sec/ml] | Pulmonary VR Resistance |
| BW | 9.7 | [kg] | Body Weight |
| BSA | 0.47 | [m²] | Body Surface Area |
| c | 20 | [ml/mg] | Gain param. FRO → SBV |

To compare the real-world direction and the analytically derived direction, the following evaluation metrics were used:

$$i ) Error {θ}_{rf} = arccos ( \frac{〈 {dy}_{r} [ k ] , {dy}_{f} [ k ] 〉}{\vert {dy}_{r} [ k ] \vert \vert {dy}_{f} [ k ] \vert} )$$

$$ii ) Error {θ}_{rp} = arccos ( \frac{〈 {dy}_{r} [ k ] , {dy}_{p} [ k ] 〉}{\vert {dy}_{r} [ k ] \vert \vert {dy}_{p} [ k ] \vert} )$$

where θ_{rf}[k] is the error angle between dyᵣ[k] (i.e., the real-world hemodynamics) and dy_{f}[k], and where θᵣₚ[k] is the error angle between dyᵣ[k] and dyₚ[k].

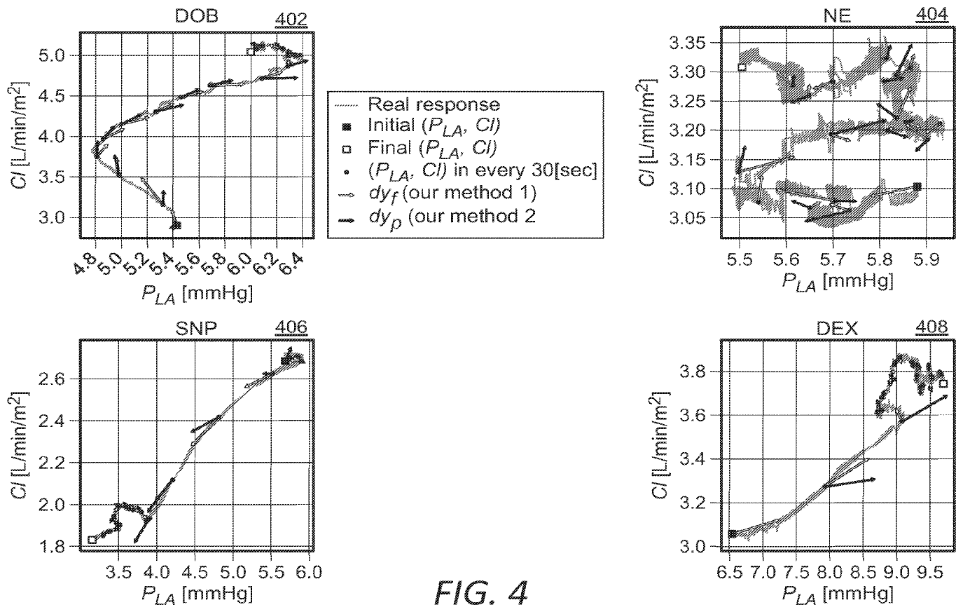

FIG. 4 depicts example charts 402-408 visualizing the hemodynamic directions of the real-world drug and the predicted response based on the analytical model, according to example embodiments of this disclosure. Particularly chart 402 shows the comparison of the real-world response and predicted response for Dobutamine (DOB), chart 404 shows the comparison of the real-world response and predicted response for Norepinephrine (NE), chart 406 shows the comparison of the real-world response and the predicted response for Sodium Nitroprusside (SNP), and chart 408 shows a comparison of the real world response and predicted response for Dextran (DEX). All the charts 402, 404, 406, and 408 show the directions of hemodynamic change at every 30 seconds. As shown, both the methods dy_{f}[k] and dyᵣ[k] can accurately predict the hemodynamic direction at each time step k.

For the angular error comparison, Table III shows the results of the angular errors comparing the real-world response and the response based on the analytical models (via using the total derivates). The shown values are average values and confidence intervals (CIs). On the average of 4 drug infusion experiments, average error θ_{rf} resulted in 18.85 degrees. In the practical prediction task when the future information is not available, the average error θᵣₚ resulted in 45.5 degrees.

TABLE III
Results of Exp. 1 Angular Errors (Real vs Ours)

| | i) Error θ_{rf} | | ii) Error θᵣₚ | |
|---|---|---|---|---|
| Drug | Error avg. [°] | C.I. [°] | Error avg. [°] | C.I. [°] |
| DOB | 16.1 | [10.4, 21.9] | 38.0 | [19.7, 56.4] |
| NE | 28.1 | [9.7, 46.4] | 57.2 | [30.7, 83.8] |
| SNP | 14.09 | [9.1, 18.9] | 39.3 | [22.5, 56.1] |
| DEX | 17.2 | [10.8, 23.7] | 47.6 | [27.3, 68.0] |

Further simulations were conducted to evaluate the disclosed analytical system, comprising the drug infusion model x=ƒ(u) with the identified drug library and the hemodynamics analytical solution y=h(x)=h(ƒ(u)) and the direction of its change dy. For example, three simulation studies were conducted to evaluate the analytical model in both qualitative and quantitative manner. The three simulations included: 1) single drug infusion, 2) three acute heart failure scenarios, and 3) recommended drug therapies via Forrester classification. All the three simulations are described in detail below.
1. Single Drug Infusion: The single drug infusion simulation experiment generally visualization how single drug infusion uᵢ affects four-dimensional hemodynamics y (i.e., P_{LA}, CO, MAP, MVO₂) with the direction of the change of the single drug infusion. The infused single dose is set as u₁=2 μg/kg/min for DOB, u₂=0.15 μg/kg/min for NE, u₃=2 μg/kg/min for SNP, u₄=75 ml for DEX, and u₅=5 mg for FRO. For the simulated acute heart failure patients, the difference combinations of the cardiovascular parameters were formed by Rₛ=[1.0, 3.0, 5.0], Eₑₛ=[6.0, 12.0], HR=[60, 120], and SBV=[100:150:700].

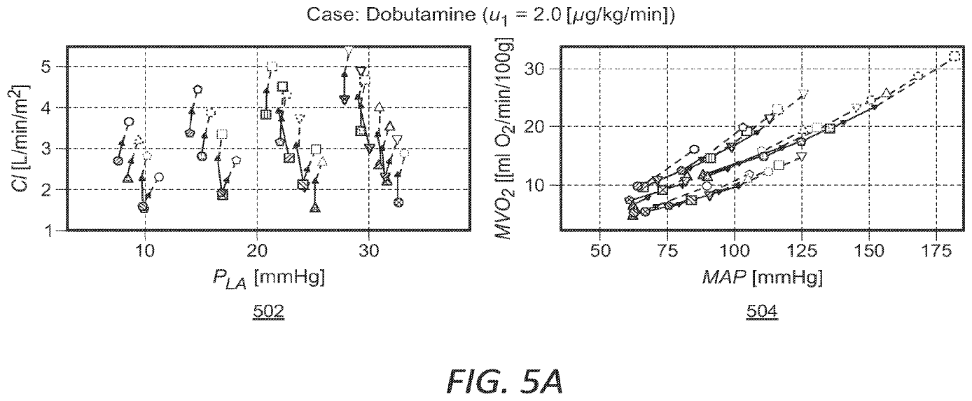

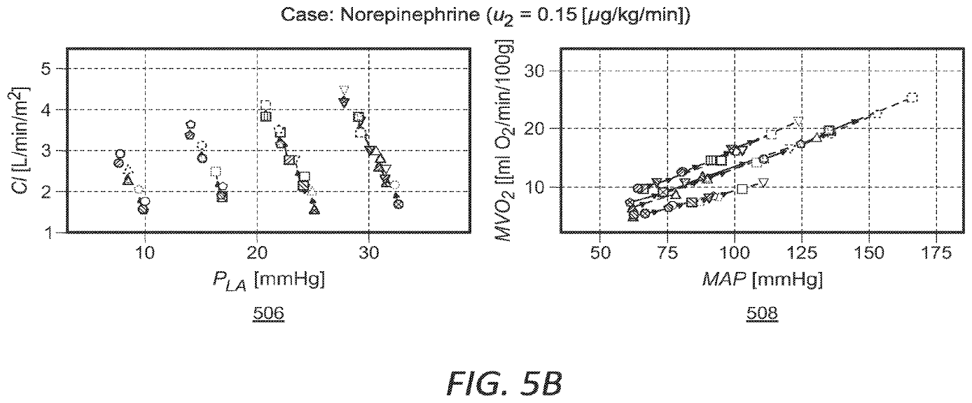

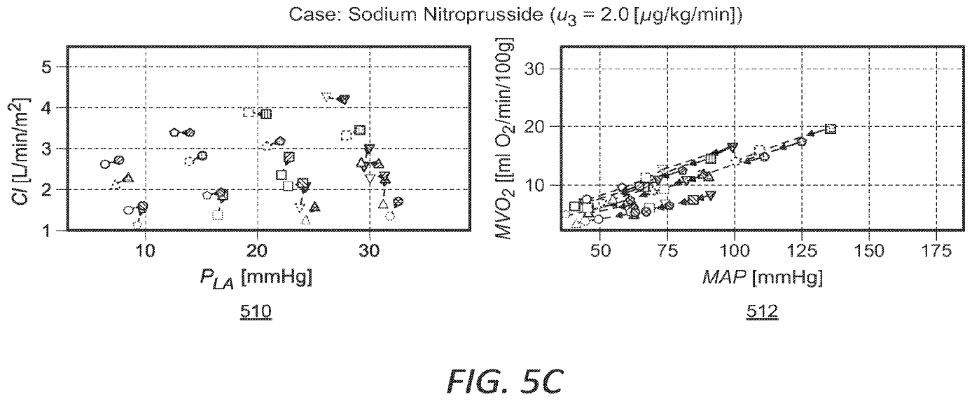

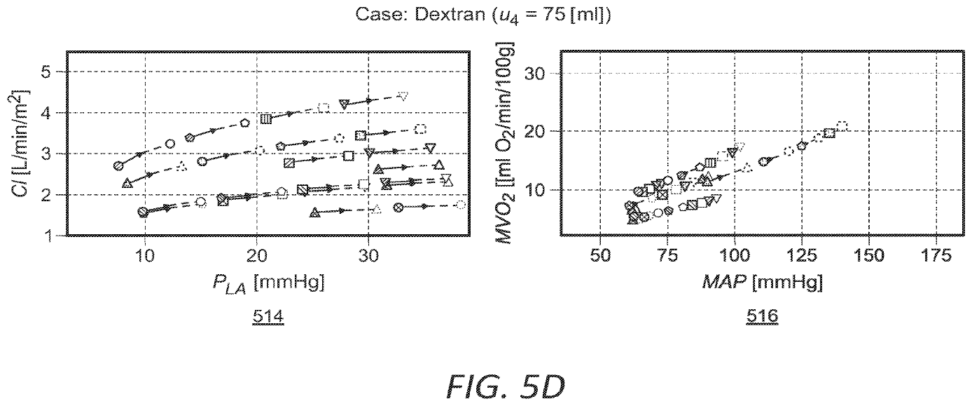

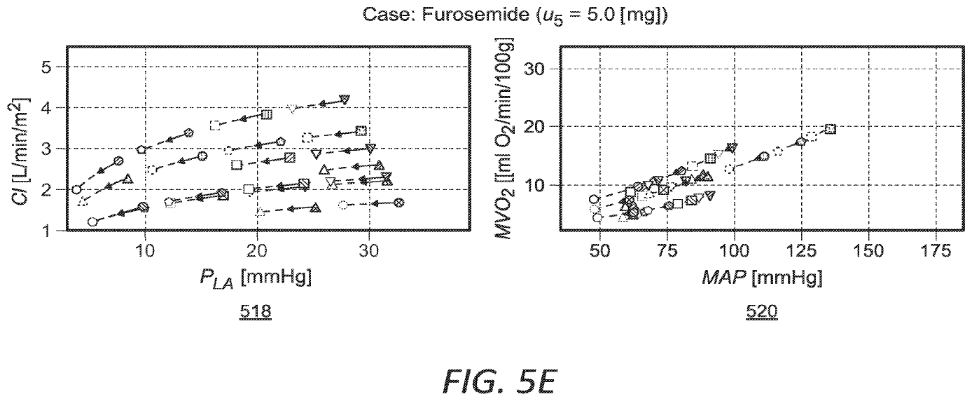

FIGS. 5A-5E depict example charts 502-520 visualizing a single drug infusion simulation, based on the example embodiments of this disclosure. As shown in the charts 502-520, the cardiovascular metrics (e.g., cardiovascular metrics 212) are divided into two spaces: (i) LAP-CI (Forrester classification) and (ii) MAP-MVO₂. In each of the charts 502-520, each filled marker shows the initial cardiovascular metrics (consistent with the visualization shown in FIG. 2) y₀ of different acute heart failure patients, the arrow shows the direction of the hemodynamic change dy, and the dashed line implies the estimated time-transient response using Bezier curve. Each empty marker shows the final outcome in steady-state y_{f} (also consistent with the visualization shown in FIG. 2).

As shown in the charts 502, 504, 506, and 508, positive inotropes (Dobutamine, Norepinephrine) improve CI and MAP but significantly increase MVO₂. As shown in charts 510, 512, 518, and 520, Sodium Nitroprusside and Furosemide decrease both P_{LA} and MVO₂. As shown in charts 514 and 516, Dextran increases both CI and P_{LA}. The visualizations provide by charts 502-520 may provide meaningful insights for clinicians because P_{LA} and MAP are measurable in a clinical setting, but MVO₂ is not directly measurable.
2. Three acute heart failure scenarios: This simulation generally visualizes three specific clinical scenarios in subset II, III, and IV on Forrester classification. Three representative patients with the following cardiovascular parameters were considered.

$$( warm and wet ) {x}_{II} = {[ 3.0 , 15 , 80 , 600 ]}^{T}$$

$$( cold and dry ) {x}_{III} = {[ 4. , 10 , 100 , 200 ]}^{T}$$

$$( cold and wet ) {x}_{IV} = {[ 8. , 8. , 100 , 450 ]}^{T}$$

Then, each drug infusion was simulated. The dosage was set to be the same amount mentioned in the single drug infusion simulation described above.

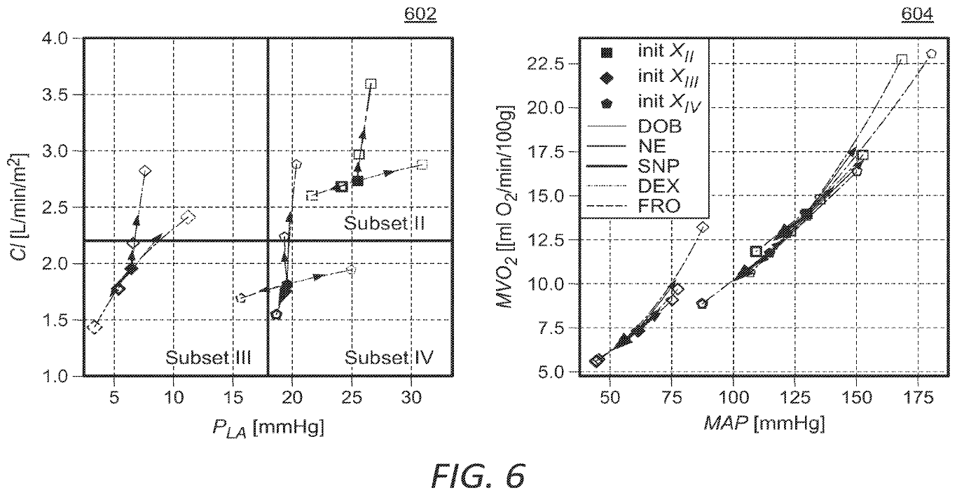

FIG. 6 depicts example charts 602-604 visualizing three acute heart failure scenarios simulation, based on example embodiments of this disclosure. In both example charts 602 and 604, the initial direction and the time transient response are consistent with the expectation of pharmacological effects in general. It is further shown that even with the same drug and same dosage, there may be difference in sensitivity depending on the patient's condition. For example, the DOB administration with the dosage of 2.0 μg/kg/min may improve CI in all scenarios and the change of MVO₂ in subset IV is notably more significant than in other scenarios as shown below:

$$( warm and wet ) {x}_{II} = + 8 \text{.88} ml {O}_{2} / min / 100 g$$

$$( cold and dry ) {x}_{III} = + 5 \text{.87} ml {O}_{2} / min / 100 g$$

$$( cold and wet ) {x}_{IV} = + 11.3 ml {O}_{2} / min / 100 g$$

The mechanism of this non-linear behavior caused by the gradients of the MVO₂ (x) shown in the Jacobian matrix above. This non-linear behavior indicates that the same dosage of DOB results in greater oxygen consumption in the heart of Subset IV patients than other patients.
3. Recommended drug therapies via Forrester classification: This third simulation was designed to validate whether the analytically predicted direction of hemodynamic change is oriented toward a desired range in (P_{LA}, CI) space when the recommended drug therapies are applied for various acute heart failure patients. For the simulated acute heart failure patients, the different combinations of cardiovascular parameters were formed by Rₛ−[1.0:1.0:5.0], Eₑₛ−[3.0:2.0:15.0], HR−[60:20:150], and SBV-[100:50:700]. Unrealistic acute heart failure patients with MAP(x)≤44 mmHg or 170 mmHg≤MAP(x) were excluded. With all the selections and exclusions, 963 different acute heart failure patients were simulated. To treat these simulated acute heart failure patients, drug combinations and the dosages were selected based on the guidelines in the Forrester classification.

$$Subset II : {u}_{II} = {[ 0. , 0. , 5. , 0. , 1.5 ]}^{T}$$

$$Subset III : {u}_{III} - {[ 5. , 0.15 , 0. , 50. , 0 ]}^{T}$$

$$Subset II : {u}_{IV} = {[ 5. , 0. , 5. , 0. , 1.5 ]}^{T}$$

Here, the metric for evaluation is whether the extension of the predicted direction from the initial state (e.g., initial state 206 in FIG. 2) belongs to a target area that is set within the normal range of normal range of Subset I: 3.0≤P_{LA}≤17.0 mmHg and 2.25≤CI≤4.5 L/min/m².

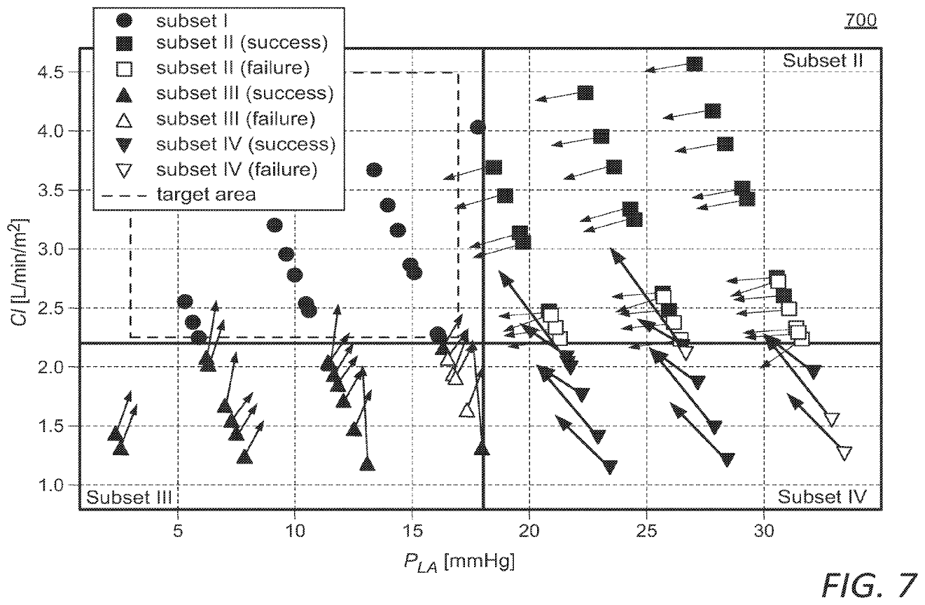

FIG. 7 depicts an example chart 700 visualizing simulation using Forrester classification, according to example embodiments of this disclosure. The chart shows that out of 963 heart failure patients, 779 patients were successfully oriented toward the target area (i.e., 80.9% success rate). This high success rate was achieved even though the drug inputs were simply fixed for each subset. Chart 700 generally shows that that the analytically derived and simulated directional orientation mostly corresponds to the expected results in the clinical guideline. Therefore, one having ordinary skill in the art will understand that the analytical models disclosed herein, including the identified drug library B and the corresponding analytical solutions, are reasonably correct.

Embodiments disclosed herein therefore allow a simultaneous visualization of how each drug affects cardiac metabolism (as indicated by MVO₂) and the circulatory system (as indicated by CI, MAP, P_{LA}). For instance, positive inotropes such as catechoolamines maintain adequate hemodynamic stability and prevent hyperfusion or pulmonary congestion. However, an increase in their dosage can cause poorer prognosis for patients with cardiogenic shock because these drugs cause metabolic stress and arrhythmogenesis in a depressed heart. This is just but an example, and it is very difficult to determine the optimal dosage of drug in a clinical setting.

As a solution to this problem, embodiments disclosed herein provide systems, methods, and devices to assist clinicians to clinical decision making regarding the optimal combination of drugs. For instance, as described above, effects of various drugs on the whole body—and the heart—can be visualized. Furthermore, embodiments disclosed herein also reveal the variation in sensitivity toward the drugs depending upon the patient's cardiovascular parameters, even if the dosage is the same. The disclosed embodiment of using the gradient analysis (e.g., by using the Jacobian matrix above) allows a quantification of immediate change due to the drugs, providing a real-time clinical decision support.

Furthermore, as described above, the combination of drugs disclosed herein is just but an example and should not be considered limiting. That is, embodiments disclosed herein can be applied to any kind of drugs. For example, the embodiments apply to beta-blockers at least because an early administration of beta-blockers has been reported to improve prognosis and to prevent sudden death resulting from arrhythmias. Furthermore, the use of a pure bradycardiac agent (Ivabradine) with conventional drugs has been reported to improve the performance of simultaneous four-dimensional hemodynamics control in dogs with acute heart failure. Embodiments disclosed herein therefore are equally applicable to any kind of drug used to improve prognosis of patients suffering from acute heart failure.

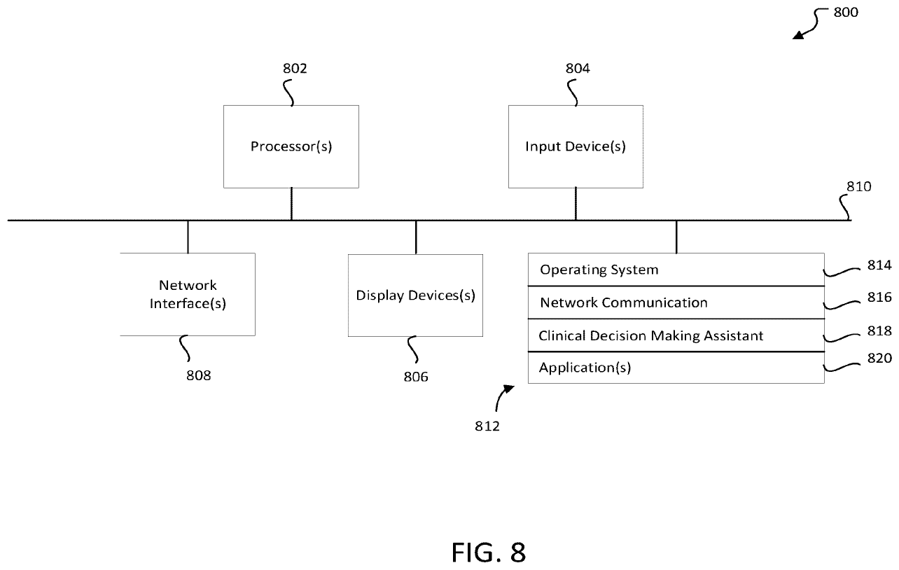

FIG. 8 shows a block diagram of an example computing device 800 that implements various features and processes, according to example embodiments of this disclosure. For example, computing device 800 may function as the server 102 and clients 106, or a portion or combination thereof in some embodiments. Additionally, the computing device 800 may partially or wholly host and deploy analytical model 200. The computing device 800 may also perform one or more steps of the methods 300. The computing device 800 is implemented on any electronic device that runs software applications derived from compiled instructions, including without limitation personal computers, servers, smart phones, media players, electronic tablets, game consoles, email devices, etc. In some implementations, the computing device 800 includes one or more processors 802, one or more input devices 804, one or more display devices 806, one or more network interfaces 808, and one or more computer-readable media 812. Each of these components is be coupled by a bus 810.

Display device 806 includes any display technology, including but not limited to display devices using Liquid Crystal Display (LCD) or Light Emitting Diode (LED) technology. Processor(s) 802 uses any processor technology, including but not limited to graphics processors and multi-core processors. Input device 804 includes any known input device technology, including but not limited to a keyboard (including a virtual keyboard), mouse, track ball, and touch-sensitive pad or display. Bus 810 includes any internal or external bus technology, including but not limited to ISA, EISA, PCI, PCI Express, USB, Serial ATA or FireWire. Computer-readable medium 812 includes any non-transitory computer readable medium that provides instructions to processor(s) 802 for execution, including without limitation, non-volatile storage media (e.g., optical disks, magnetic disks, flash drives, etc.), or volatile media (e.g., SDRAM, ROM, etc.).

Computer-readable medium 812 includes various instructions 814 for implementing an operating system (e.g., Mac OS®, Windows®, Linux). The operating system may be multi-user, multiprocessing, multitasking, multithreading, real-time, and the like. The operating system performs basic tasks, including but not limited to: recognizing input from input device 804; sending output to display device 806; keeping track of files and directories on computer-readable medium 812; controlling peripheral devices (e.g., disk drives, printers, etc.) which can be controlled directly or through an I/O controller; and managing traffic on bus 810. Network communications instructions 816 establish and maintain network connections (e.g., software for implementing communication protocols, such as TCP/IP, HTTP, Ethernet, telephony, etc.).

Clinical decision-making assistant 818 includes instructions that implement the disclosed process for assisting clinical decision making, as described throughout this disclosure. Application(s) 820 may comprise an application that uses or implements the processes described herein and/or other processes. The processes may also be implemented in the operating system.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment. In one embodiment, this may include Python. The computer programs therefore are polyglots.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

Additional examples of the presently described method and device embodiments are suggested according to the structures and techniques described herein. Other non-limiting examples may be configured to operate separately or can be combined in any permutation or combination with any one or more of the other examples provided above or throughout the present disclosure.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to.” If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A system comprising:
a non-transitory storage medium storing computer program instructions; and
one or more processors configured to execute the computer program instructions to cause operations comprising:
receiving a plurality of current cardiovascular metrics for a patient with acute heart failure;
executing a clinical decision making assistance module on the plurality of current cardiovascular metrics to generate a recommended combination of drugs to reach a plurality of target cardiovascular metrics, the clinical decision making assistance module being based on:
a drug library linearly modeling a pathway from a plurality of current cardiovascular parameters associated with the plurality of current cardiovascular metrics to a plurality of target cardiovascular parameters associated with the plurality of target cardiovascular metrics; and
a mapping between the plurality of target cardiovascular metrics and the plurality of target cardiovascular parameters, the mapping including a time transient directional response of one or more cardiovascular metrics due to a change in one or more cardiovascular parameters caused by an administration of one or more drugs; and
outputting the recommended combination of drugs.

2. The system of claim 1, the plurality of current cardiovascular metrics and the plurality of target cardiovascular metrics comprising one or more of left atrial pressure, cardiac output, mean arterial pressure, or myocardial oxygen consumption.

3. The system of claim 1, the plurality of current cardiovascular parameters and the plurality of target cardiovascular parameters comprising one or more of systemic vascular resistance, cardiac contractility, heart rate, or stressed blood volume.

4. The system of claim 1, the one or more drugs comprising at least one of positive inotropes, vasopressors, vasodilators, fluids, or diuretics.

5. The system of claim 1, the plurality of current cardiovascular metrics and the plurality of target cardiovascular metrics comprising myocardial oxygen consumption.

6. The system of claim 1, the plurality of current cardiovascular metrics and the plurality of target cardiovascular metrics comprising myocardial oxygen consumption being represented as a function of one or more cardiovascular metrics.

7. The system of claim 1, the mapping further comprising a sensitivity of the time transient directional response of the one or more cardiovascular metrics due to the change in the one or more cardiovascular parameters caused by the administration of the one or more drugs.

8. The system of claim 7, the mapping comprising a Jacobian matrix.

9. The system of claim 8, wherein columns of the Jacobian matrix indicate the sensitivity of a change in a single cardiovascular parameter affecting multiple cardiovascular metrics.

10. The system of claim 8, wherein rows of the Jacobian matrix indicate the sensitivity of a change in multiple cardiovascular parameters affecting a single cardiovascular metric.

11. A computer-implemented method comprising:
receiving, from a client device by a computing system, a plurality of current cardiovascular metrics for a patient with acute heart failure;
executing, by the computing system, a clinical decision making assistance module on the plurality of current cardiovascular metrics to generate a recommended combination of drugs to reach a plurality of target cardiovascular metrics, the clinical decision making assistance module being based on:
a drug library linearly modeling a pathway from a plurality of current cardiovascular parameters associated with the plurality of current cardiovascular metrics to a plurality of target cardiovascular parameters associated with the plurality of target cardiovascular metrics; and
a mapping between the plurality of target cardiovascular metrics and the plurality of target cardiovascular parameters, the mapping including a time transient directional response of one or more cardiovascular metrics due to a change in one or more cardiovascular parameters caused by an administration of one or more drugs; and
outputting, by the computing system to the client device, the recommended combination of drugs.

12. The computer-implemented method of claim 11, the plurality of current cardiovascular metrics and the plurality of target cardiovascular metrics comprising one or more of left atrial pressure, cardiac output, mean arterial pressure, or myocardial oxygen consumption.

13. The computer-implemented method of claim 11, the plurality of current cardiovascular parameters and the plurality of target cardiovascular parameters comprising one or more of systemic vascular resistance, cardiac contractility, heart rate, or stressed blood volume.

14. The computer-implemented method of claim 11, the one or more drugs comprising at least one of positive inotropes, vasopressors, vasodilators, fluids, or diuretics.

15. The computer-implemented method of claim 11, the plurality of current cardiovascular metrics and the plurality of target cardiovascular metrics comprising myocardial oxygen consumption.

16. The computer-implemented method of claim 11, the plurality of current cardiovascular metrics and the plurality of target cardiovascular metrics comprising myocardial oxygen consumption being represented as a function of one or more cardiovascular metrics.

17. The computer-implemented method of claim 11, the mapping further including a sensitivity of the time transient directional response of the one or more cardiovascular metrics due to the change in the one or more cardiovascular parameters caused by the administration of the one or more drugs.

18. The computer-implemented method of claim 17, the mapping comprising a Jacobian matrix.

19. The computer-implemented method of claim 18, wherein columns of the Jacobian matrix indicate the sensitivity of a change in a single cardiovascular parameter affecting multiple cardiovascular metrics, and wherein rows of the Jacobian matrix indicate the sensitivity of a change in multiple cardiovascular parameters affecting a single cardiovascular metric.

20. A non-transitory storage medium storing computer program instructions, which when executed by one or more processors cause operations comprising:
receiving a plurality of current cardiovascular metrics for a patient with acute heart failure;
executing a clinical decision making assistance module on the plurality of current cardiovascular metrics to generate a recommended combination of drugs to reach a plurality of target cardiovascular metrics, the clinical decision making assistance module being based on:
a drug library linearly modeling a pathway from a plurality of current cardiovascular parameters associated with the plurality of current cardiovascular metrics to a plurality of target cardiovascular parameters associated with the plurality of target cardiovascular metrics; and
a mapping between the plurality of target cardiovascular metrics and the plurality of target cardiovascular parameters, the mapping including a time transient directional response of one or more cardiovascular metrics due to a change in one or more cardiovascular parameters caused by an administration of one or more drugs; and outputting the recommended combination of drugs.
